(19) United States
(12) Patent Application Publication
(10) Pub. No.: US 2013/0260391 A1
(43) Pub. Date: Oct. 3, 2013

(54) Method Of Determining The Protease Cathepsin B In A Biological Sample

(71) Applicants: Hans Jörg Meier, Konstanz (DE); Hanns Jörg Mauk, Mundelsheim (DE)

(72) Inventors: Hans Jörg Meier, Konstanz (DE); Hanns Jörg Mauk, Mundelsheim (DE)

(21) Appl. No.: 13/807,619

(22) PCT Filed: Jul. 1, 2011
(86) PCT No.: PCT/EP11/61138

§ 371 Date: Jun. 17, 2013

(30) Foreign Application Priority Data
Jul. 2, 2010 (DE) 10 2010 025 970.5

(51) Int. Cl. G01N 33/573

(52) U.S. Cl. CPC G01N 33/573
USPC 435/7.4

(57) ABSTRACT

A method for determination of the potentially available activity of cathepsin B in a biological sample, including the activity of the active form of cathepsin B, the form of cathepsin B which can be activated from the pro-form procathepsin B being present in the sample, and the form of cathepsin B which can be activated and which is inhibited in the sample in its activity by protease inhibitor, whereby the procathepsin B being present in the sample is converted into the active form of cathepsin B, the free protease inhibitor for cathepsin B being present in the sample is depleted from the sample or its inhibitor function is suppressed, and the protease inhibitor is withdrawn from the inhibited form of cathepsin B, and subsequently the activity of the active cathepsin B in the sample is determined.

### SUMMARY OF THE INVENTION

The invention relates to a method for determination of the potentially available activity of cathepsin B in a biological sample, including the activity of the active form of cathepsin B, and the form of cathepsin B which can be activated from the pro-form procathepsin B being present in the sample, and the form of cathepsin B which can be activated and which is inhibited in the sample in its protease activity by a protease inhibitor.

### BACKGROUND OF THE INVENTION

For the determination of the concentration of enzymes in tissue extracts or in body fluids, e.g. in serum, enzyme assays and immunological tests, e.g. ELISA, are available. In case of medical analytics and clinical diagnostics it is often important to determine the present amount of intact and active enzyme in a sample, so as to be able to make statements on the aetiology, on the effects of the disease and on the prognosis. Permanently inactive forms of enzymes, e.g. denaturated enzymes or fragments of enzymes, are often irrelevant and therefore are not taken into account in the determination of such enzymes and often should not be taken into account.

Other forms of inactive enzymes such as enzymes the activity of which are temporarily and reversibly inhibited by inhibitors or enzyme precursors, so called proenzymes, however may play a decisive rule as to causes, effects and/or prognoses of diseases. Such forms of inactive enzymes may be converted to their active forms in certain circumstances, if their enzyme activity is required for biological processes. In such cases the inhibitors, for example, are inactivated by certain mechanisms or withdrawn from the enzyme, or proenzymes are converted in their active forms. The overall concentration of active and temporarily inactive enzymes in tissue or body fluids may give important hints for specific diseases.

Enzyme assays for determination of the concentration of enzymes employ such reactions which are carried out or catalysed by the active enzymes. If, however, also such enzyme forms are to be included which are available in the biological sample as temporarily inhibited inactive forms and/or as inactive proenzymes, which is, for example, often the case with proteases, then these enzyme forms cannot be measured in such assays due to their inactivity. Therefore the enzyme activity measurable in a sample does often not correspond with the really available enzyme activity, because at least a part of the enzyme activity is inhibited by inhibitors or is present in the inactive form of the proenzyme.

It is a well-known fact that in tissue samples of cancer patients infested with cancer the level of the lysosomal cysteine protease cathepsin B is increased compared with the corresponding tissue samples of healthy persons. Therefore cathepsin B is considered as a diagnostic and prognostic factor in cancer diseases. There a part of cathepsin B is in a cystatin-inhibited form. The cysteine protease inhibitors to be considered in this case belong to the superfamily of the cystatines, and cystatin A and cystatin B from the family I are intracellularly active, and cystatin C from the family II is extracellularly active, thus also in the serum.

In WO 97/00969, for the enzymatic determination of the concentration of cathepsin B it is proposed in tissue samples to withdraw the cystatine inhibitors from the inhibited cathepsin B by affinity chromatography, whereby a biological sample is passed through an affinity chromatography column filled with sepharose gel to which papain is covalently bound. Papain is also a cysteine protease and has a higher binding affinity to cystatines then the cathepsins, and therefore papain withdraws the inhibitor from cathepsin. Afterwards the activity of the deinhibited cathepsin B is measured by utilising its enzymatic activity.

Unlike tissue samples, in serum it was observed that without deinhibition an activity of cathepsin B cannot be measured. Therefore, it is assumed that in serum the whole cathepsin B is inhibited.

Measurements by direct ELISA have shown that in the serum of prostate cancer patients the cumulative value of procathepsin B, the proenzyme of cathepsin B, and of cathepsin B is about threefold increased as compared with the corresponding value of healthy persons, while at the same time the activity of cathepsin B in the serum which was deinhibited before the measurement was only about 30% higher as compared with healthy persons. And by means of a sandwich-ELISA the cumulative value of the concentrations of cathepsin B and procathepsin B in serum of colon cancer patients was increased about fivefold as compared with healthy persons. Therefore, it is assumed that the concentration of procathepsin B or the cumulative value of the concentration of cathepsin B and procathepsin B is a more significant indicator for cancer diseases than the value of cathepsin B alone.

In immunological assays such as ELISA the enzyme molecules in a sample are specifically detected by means of antibodies. Though the immunological assay is generally more sensitive than the enzymatic assay, the antibodies do not discern between the active form of the enzyme and the form of the enzyme which is inhibited by inhibitors; thus these immunological assays detect the cumulative amount of active and inhibited enzyme. According to the antibody used in such immunological assays also proenzymes, permanently inactive enzymes and to some extent also denatured enzymes can be detected. As the assessment of the causes, the implications and the prognosis of a disease on the basis of enzymes depends usually on the enzymes which are active or can be activated, because only these forms of the enzyme eventually trigger or catalyse biological processes, such immunological assays which also detect permanently inactive forms of enzymes may negatively affect the desired significance of the used assay and are therefore only partly qualified for medical analytics and diagnosis. In addition immunological assays often require high expenditures of equipment and time and are therefore cost-intensive and normally can be carried out in the medical sector only in special labs.

### TASK OF THE INVENTION

The task of the present invention is therefore to provide a method for the determination of the protease cathepsin B which is especially relevant for cancer diagnosis, a method being more cost-effective and simpler to perform as compared to well-known immunological methods and at the same time being suitable to determine in a biological sample, particularly in blood plasma or serum, active cathepsin B, by inhibitors reversibly inhibited cathepsin B, and procathepsin B, and to avoid errors by the detection of permanently inactive cathepsin B.

### DESCRIPTION OF THE INVENTION

The task is solved by means of a method for determination of the potentially available activity of cathepsin B in a biological sample, including the activity of the active form of cathepsin B, and the form of cathepsin B which can be activated from the pro-form procathepsin B being present in the sample, and the form of cathepsin B which can be activated and which is inhibited in the sample in its activity by protease inhibitors, with the following steps:

a) procathepsin B being present in the sample is converted into the active form of cathepsin B by means of
a.i) contacting the sample with a first enzyme (proteolytic enzyme) the functionality of which is able to convert procathepsin B into the active form of cathepsin B by proteolytic digestion, or
a.ii) lowering the pH value to a value where procathepsin B is converted into the active form of cathepsin B,

b) depletion of the free protease inhibitor of cathepsin B in the sample or suppression of its inhibitor function and withdrawing the protease inhibitor from the inhibited form of cathepsin B by contacting the sample with a second enzyme (inhibitor binding enzyme) which is able to bind the protease inhibitor of cathepsin B and has a higher affinity to the protease inhibitor than cathepsin B, whereby
b.i) the first enzyme (proteolytic enzyme) and the second enzyme (inhibitor binding enzyme) are the same enzyme provided that the enzyme has the function to convert procathepsin B into the active form of cathepsin B by proteolytic digestion as well as to bind the protease inhibitor for cathepsin B and has a higher affinity to the protease inhibitor than cathepsin B, or
b.ii) the utilised second enzyme (inhibitor binding enzyme) differs from the first enzyme (proteolytic enzyme), if it is used,
   whereby

the second enzyme (inhibitor binding enzyme) is an enzyme which has not a proteolytic activity for degrading cathepsin B, or

the second enzyme (inhibitor binding enzyme) is an enzyme which has a proteolytic activity for degrading cathepsin B, and this proteolytic activity of the second enzyme for degrading cathepsin B is inactivated after a reaction time of the step b) in the sample, or the second enzyme is removed from the sample and substituted by an enzyme which has not an activity for degrading cathepsin B but is able to bind the protease inhibitor for cathepsin B and has a higher affinity to the protease inhibitor than cathepsin B,

c) contacting the sample with a substrate for cathepsin B and recording the proteolytic reaction of the substrate catalysed by the protease cathepsin B.

In the present invention the term “potentially available activity” of cathepsin B in a biological sample denotes the activity of cathepsin B being available if in the presence of the active form of cathepsin B present in the sample the temporarily inactive forms of cathepsin B, such as procathepsin B and reversibly inhibited forms of cathepsin B are converted into the active forms.

The inventive method for determination the potentially available activity of cathepsin B in a biological sample is based on the fact that all active forms of cathepsin B in the biological sample as well as the forms which can be activated, i.e. the active form of cathepsin B, the forms of cathepsin B being reversibly inhibited by protease inhibitors, and the biological precursor procathepsin B, at first are converted into an active form and then an enzymatic reaction is carried out which is specific for cathepsin B with a substrate which is specific for cathepsin B.

Therefore in a first step a) of the inventive method procathepsin B is converted into the active form of cathepsin B. Preferably this is done enzymatically, whereby the sample is brought into contact with a first enzyme (proteolytic enzyme) which has the functionality to convert procathepsin B into the active form of cathepsin B by proteolytic digestion. Alternatively, procathepsin in the sample can also be converted into the active form of cathepsin B by lowering the pH value. In doing so, the sample, which has usually a physiological pH value in the range between 6 and 8, has to be set to a lower pH value in the range between 3.5 and 5.5, preferably in the range between 4.0 and 5.0, particularly preferred at about 4.5 where the pro-region of procathepsin B is cleaved.

In a preferred embodiment of the previous inventive method the first enzyme (proteolytic enzyme) which has the functionality to convert procathepsin B into the active form of cathepsin B by means of proteolytic digestion and which is brought into contact with the sample, is a hydrolase which lacks the proteolytic activity for degrading cathepsin B, and which is preferably pepsin or cathepsin D or cathepsin C or thermolysin or pronase, particularly preferred pepsin or cathepsin D. In this embodiment the utilized second enzyme (inhibitor binding enzyme) differs from the first enzyme (proteolytic enzyme), as the above mentioned hydrolases usually have not the functionality to bind the protease inhibitor for cathepsin B. Pepsin is particularly preferred as the first enzyme. Though the previously listed hydrolases are capable to proteolytically cleave the pro-region of procathepsin B and thus convert procathepsin B into cathepsin B, the product cathepsin B will not significantly be digested further by pepsin or cathepsin D.

In a first variant of this method in which the first enzyme (proteolytic enzyme) is a hydrolase without protease activity for degrading cathepsin B, but has the functionality to convert procathepsin B into the active form of cathepsin B by proteolytic digestion, the utilised second enzyme (inhibitor binding enzyme) is papain which has the functionality to bind the protease inhibitor for cathepsin B and has a higher affinity to the protease inhibitor than cathepsin B, whereby the protease activity of papain for degrading cathepsin B is inactivated in the sample after a reaction time or the papain is removed from the sample and substituted by an enzyme which has not a protease activity for degrading cathepsin B, but has the functionality to bind the protease inhibitor for cathepsin B and has a higher affinity to the protease inhibitor than cathepsin B.

In a second variant of the method where the first enzyme (proteolytic enzyme) is a hydrolase without a protease activity for degrading cathepsin B, but has the functionality to convert procathepsin B into the active form of cathepsin B by proteolytic digestion, the utilized second enzyme (inhibitor binding enzyme) is a modified papain which has the functionality to bind the protease inhibitor for cathepsin B and has a higher affinity to the protease inhibitor than cathepsin B, but lacks the functionality to convert procathepsin B into the active form of cathepsin B by proteolytic digestion and the protease activity for degrading cathepsin B, whereby the modified papain lacking the protease activity can be made by

a) chemical modification of the SH-group of cysteine in the proteolytic active centre of papain, preferably by reaction of papain with methyl methanthiosulfonate (MMTS), p-mercuribenzoate or AgNO₃ or by addition of N-substituted maleinimide, or by

b) site-directed mutation of the cysteine in the proteolytically active centre of papain by another amino acid.

In the inventive “modified” papain the proteolytic activity for the degradation of cathepsin B is switched off, however it still has the papain inherent high affinity for binding the cystatine protease inhibitors.

The modified papain lacking the protease activity can be made chemically, what can also be used for in situ modification of papain in the sample so as to switch off its protease activity against cathepsin B only after a reaction time when its proteolytic activity has converted procathepsin B into cathepsin B. A preferred chemical method for inactivating the protease activity of papain is the oxidation of the SH-group of cysteine in the proteolytic active centre of papain. In a particularly preferred embodiment of the invention this chemical oxidation of the SH-group is carried out by reaction of papain with methyl methanthiosulfonate (MMTS). Alternatively, the inactivation of the protease activity of papain can be achieved by masking the SH-group with heavy metal compounds such as p-mercuribenzoate or AgNO₃, or by addition of N-substituted maleinimide to the SH-group.

In an alternative embodiment of the invention the modified papain having an inactivated protease activity can be made by exchange or site-directed mutation of the cysteine in the proteolytic active centre of papain by another amino acid. The genetic engineering methods of cloning for such an exchange or the production of a site-directed mutation of the cysteine residue are known to the person skilled in the art as well as the subsequent production of the papain mutant by means of expression in vitro or in vivo and subsequent isolation or purification.

In an alternative embodiment of the inventive method the utilised first enzyme (proteolytic enzyme) and the second enzyme (inhibitor binding enzyme) are the same enzyme, namely papain, which has the functionality to convert procathepsin B into the active form of cathepsin B by means of proteolytic digestion as well as to bind the protease inhibitor for cathepsin B and has a higher affinity to the protease inhibitor than cathepsin B, whereby the protease activity of papain for degrading cathepsin B is inactivated after a reaction time or the papain is removed from the sample and substituted by an enzyme which lacks the protease activity for degrading cathepsin B, but has the functionality to bind the protease inhibitor for cathepsin B and has a higher affinity to the protease inhibitor than cathepsin B.

Papain is able to digest cathepsin B, which has been set free in the sample, and thus can deprive active cathepsin B from the sample. It is assumed that under optimal reaction conditions active papain degrades about 4 to 5% of cathepsin B during about five minutes. This is not the case for pepsin or cathepsin D. The degradation of cathepsin B by papain may negatively affect the measuring results, as a smaller amount of active cathepsin B is recorded as compared to the amount which was actually available in the biological sample. Therefore, papain has to be removed after a reaction time from the sample or its protease activity against the degradation of cathepsin B has to be inactivated. Embodiments for this purpose of the inventive method are described in the following.

In a preferred embodiment of the inventive method, where papain is utilized as the first and the second enzyme or only as the second enzyme (inhibitor binding enzyme), after a reaction time the papain in the sample is transformed into a modified papain having an inactivated protease activity for degradation of cathepsin B, whereby the modified papain having an inactivated protease activity can be made by chemical modification of the SH-group of the cysteine in the proteolytic active centre of papain, preferably by reaction of papain with methyl methanthiosulfonate (MMTS), p-mercuribenzoate or AgNO₃ or by addition of N-substituted maleinimide.

In this manner papain remains his functionality for withdrawing or binding the protease inhibitors, but looses its protease activity, so that after inactivating the papain, i.e. after converting papain into its modified form, the reaction batch can be further processed and the time course thereof does not become critical at least in regard to the proteolytic degradation of cathepsin B.

In an alternatively preferred embodiment of the inventive method, in which papain is utilized as the first and the second enzyme or only as the second enzyme (inhibitor binding enzyme), the papain in the sample is removed from the sample after a reaction time and substituted by a modified papain, which lacks the protease activity for degrading cathepsin B, but has the functionality to bind the protease inhibitor for cathepsin B and a higher affinity to the protease inhibitor than cathepsin B, whereby the modified papain lacking the protease activity can be made by

a) chemical modification of the SH-group of the cystein in the proteolytic active centre of papain, preferably by reaction of the papain with methyl methanthiosulfonate (MMTS), p-mercuribenzoate or AgNO₃ or by addition of N-substituted maleinimide, or by

b) site-directed mutation of the cysteine in the proteolytic active centre of the papain by another amino acid.

In order to remove the papain from the sample in a simple manner, the papain is preferably bound to a rigid carrier, which then at first is brought into contact with the fluid sample and after a reaction time is removed from the sample. The fluid sample may also be passed over the rigid carrier to which papain is bound so as to bring the sample into contact with the papain.

As within five minutes about 4 to 5% of the free cathepsin B is digested by completely intact papain, in an embodiment of the inventive method of the previously described variant, in which at first intact papain is brought into contact with the sample for a reaction time and then be transferred into modified papain or removed from the sample, the inactivation or removing of the protease activity is carried out after a reaction time of the deinhibition step b) of five minutes, particularly preferred after a reaction time of three minutes, exceptionally preferred after a reaction time of one minute. The faster the inactivation or removing of the active intact papain is carried out, the lesser is the degradation of cathepsin B and therewith the deviation of the measured activity of cathepsin B compared to the actually potentially available activity of cathepsin B in the biological sample.

The inventive method can advantageously carried out in blood, blood plasma, serum or in a tissue homogenate as the biological sample. Particularly preferred is the use of serum as the biological sample due to the inherent fluorescence of certain components of the blood. The inventive method may also be carried out with a tissue homogenate, however that requires a biopsy from the patient along with a surgical intervention and the processing of the tissue to a fluid sample. Serum and blood plasma are therefore particularly preferred to blood and tissue homogenate.

After the inventive conversion of procathepsin B into cathepsin B and after removing the free inhibitor for cathepsin B from the sample and the deinhibition of cathepsin B by withdrawing the protease inhibitor by means of an enzyme which has a higher affinity to the protease inhibitor than cathepsin B, the determination of activity of active cathepsin B in the sample is carried out by contacting the sample with a substrate for cathepsin B and recording the proteolytic reaction of the substrate by the protease cathepsin B. For this purpose several substrates for cathepsin B may be used. Particularly preferred is the substrate for cathepsin B that includes a di- or oligopeptide sequence and a fluorophore which can be cleaved from the oligopeptide sequence by the proteolytic reaction of the substrate by means of the protease cathepsin B, whereby the particularly preferred fluorophore is 7-amino-4-(trifluoromethyl)coumarin (AFC) or 7-amino-4-methylcoumarin (AMC), whereby AFC is particularly preferred. The fluorophore AFC or AMC is bound via the C-terminus to the di- or oligopeptide sequence. In the proteolytic reaction the fluorophore is cleaved from the di- or oligopeptide sequence. An example of a dipeptide sequence, which is specifically recognized from the cysteine protease cathepsin B and which is cleaved, is Arg-Arg. Therefore Z-Arg-Arg-AFC, for example, is suitable as a substrate, whereby Z is a protecting group, preferably an acetate-group or a carboxybenzyl-group.

The substrate for cathepsin B preferably has the characteristic feature that the uncleaved substrate, which includes the di- or oligopeptide sequence and the fluorophore, has a maximum of the fluorescence-emission wavelength which differs significantly from the maximum of the fluorescence-emission wavelength of the fluorophore, which is cleaved by the protease during the proteolytic reaction, at least by 20 nm, preferably at least by 40 nm or more. If the wavelengths or the maxima of the wavelengths of the fluorescence emission of the non-cleaved substrate and the cleaved fluorophore are identical or close to each other, then in the measurement the inherent fluorescence of the non-cleaved substrate as well as the fluorescence emission of the cleaved fluorophore is recorded. Such substrates and fluorophores having identical fluorescence-emission wavelengths are well-known in the state of art. In case of such substrates the measurement is possible in spite of the coincident fluorescence-emission wavelengths, if the intensity of the fluorescence-emission of the fluorophore is significantly higher compared with the non-cleaved substrate at the same or similar wavelength. In this case the increase of the signal is a measure for the enzyme activity as compared with an enzyme-free negative-control. A disadvantage of such substrates is, however, that a significant result can only be measured in case of a strong enzyme activity and thus of a very significant increase of the fluorescence emission, as the signal is often too weak in case of a small enzyme activity and does not differ sufficiently from the inherent fluorescence of the non-cleaved substrate. The signal gets lost in the noise or at least does not differ from it in a significant manner.

A shift of the detection wavelength of the fluorescence emission between the substrate and the cleaved fluorophore has the particular advantage that at this wavelength essentially the fluorophore which is cleaved from the substrate will only be recorded and thus only the enzymatic reaction which actually took place. The greater the distance is between the emission wavelength of the cleaved fluorophore and the fluorescence wavelength of the substrate, the more sensitive the determination of the protease activity can be.

In case of the substrate with the dipeptide Arg-Arg and the fluorophore, which is covalently bound to the C-terminus of the dipeptide, the fluorescence emission is in the blue wavelength region (ca. 460 nm), whereas the AFC fluorophore itself has a yellow-green fluorescence (ca. 505 nm). This fact guarantees a sufficient distance of the wavelengths between the non-cleaved substrate and the pure fluorophore cleaved during the enzymatic reaction. The use of the AFC fluorophore is particularly preferred compared to the AMC fluorophore, as the fluorescence emission of the substrate consisting of the dipeptide Arg-Arg and the AMC fluorophore and of the free AMC fluorophore are both in the blue wavelength region (ca. 460 nm). A discrimination between the non-cleaved substrate and the cleaved fluorophore is in this case only possible by the signal intensity but not by the emission wavelength.

In the inventive method the biological sample may be brought into contact in different ways with the enzyme which has a higher affinity to the protease inhibitor than cathepsin B and which has the functionality to remove the inhibitor for cathepsin B being free in the sample and to withdraw it from the inhibited forms of cathepsin B. In an embodiment of the inventive method the enzyme is added in its free form, advantageously solved in a buffered solution. The enzyme is spread in the sample and will bind the protease inhibitor and thus also withdraw the inhibitor from cathepsin B being in the sample. For the measurement of the concentration or activity of cathepsin B in the subsequent step of the method the enzyme may remain in the sample, if it does not disturb the specific proteolytic reaction of the substrate by the protease cathepsin B and the subsequent recording. This variant has the particular advantage that the conversion of the procathepsin B into cathepsin B and the deinhibition of the cathepsin B may be carried out in a simple manner in a single reaction vessel, for instance, in a cuvette for the subsequent measurement of the fluorescence of the subsequent enzyme assay.

In an alternative embodiment of the inventive method the enzyme, which has a higher affinity to the protease inhibitor than cathepsin B and has the functionality to remove the free inhibitor in the sample for cathepsin B and to withdraw the protease inhibitor of the inhibited forms of cathepsin B, is bound covalently or in an adsorptive manner to a rigid carrier, preferably to cellulose, particularly preferred covalently bound to cellulose, whereby the covalent binding may be obtained chemically or in a photochemical manner.

The use of a rigid carrier, to which the enzyme having a high affinity to the protease inhibitor is bound, has the advantage that this enzyme together with the protease inhibitor from the sample bound to it does not remain in the sample during the measurement of the activity and therefore does not cause any disturbance. The carrier may have any shape. For example, the carrier may be shaped as a foil or as a strip with the enzyme bound to it, which is brought into contact with the sample by dipping. After a reaction time the carrier is removed from the sample and subsequently the determination of the active cathepsin B is carried out. The dipping into the sample may be done one time or several times so as to withdraw protease inhibitor as quantitatively as possible.

The carrier may also be provided in another shape which is qualified for a tight contacting of the fluid sample with the surface of the carrier to which the enzyme is bound. For example, the carrier may be shaped as thin tubes or as capillary tubes, to the inner surface of which the enzyme is bound and through which the sample is passed. Furthermore, the carrier may be shaped as particles, beads or the like, to the inner surface of which the enzyme is bound. By dipping the particulate material into the fluid sample and if necessary by moving the particles in the sample, the enzyme is brought into tight contact with the sample and subsequently separated by centrifugation, sedimentation, filtration, or simple pipetting the fluid sample off. As described above, in a particularly preferred embodiment of the inventive method, the step a) of contacting the sample with the first enzyme having the functionality to convert procathepsin B into the active form of cathepsin B by proteolytic digestion is carried out with pepsin or cathepsin B¹ as the first enzyme. Advantageously, this step a) is carried out at a temperature in the range from 4 to 40° C., preferably between 20 and 40° C. and at a pH-value in the range between 3.5 and 5.5, preferably in the range between 4.0 and 5.0, particularly preferred at about 4.5. The enzyme pepsin has its highest proteolytic activity in the acid pH range and is essentially inactive at a pH-value above 6. Therefore it is advantageous to set the biological sample to a pH-value in the previously mentioned range or to buffer the sample, respectively, so as to provide for an optimal proteolytic digestion by pepsin or cathepsin B¹. ¹ Comment of the translator: it should read: cathepsin D instead of cathepsin B

In a further embodiment of the inventive method the subsequent step b) of contacting the sample with the second enzyme having a higher affinity to the protease inhibitor than cathepsin B and having the functionality to remove the free inhibitor in the sample and to withdraw the protease inhibitor from the inhibited forms of cathepsin B, is carried out at a temperature in the range between 4 to 40° C., preferably between 20 and 40° C. and at a pH-value in the range between 2 and 7, preferred between 4.5 and 6.

If the second enzyme performs its functionality of deinhibition under the same conditions as it is applied in the previous mentioned step a) of the cleavage of procathepsin B, then the reaction conditions regarding the temperature and the pH-value can be maintained. If a variation of the temperature and/or the pH-value is required for an optimal effect of the deinhibition by means of the second enzyme, then the reaction conditions need to be changed. A variation of the pH-value toward a value being better and optimal for the second enzyme may be achieved by addition of a suitable acid or base or of a appropriate buffer.

The invention includes also a method for the determination of the pro-form of cathepsin B, i.e. procathepsin B, in the sample, whereby

i) with a first part of the biological sample a first determination of the potentially available activity of cathepsin B is carried out according to the herein described and claimed method, whereby in the step a) of the method also the procathepsin B being present in the sample is converted into the active form of cathepsin B,

ii) with a second part of the same biological sample a second determination of the potentially available activity of cathepsin B is carried out according to the herein described and claimed method, whereby the second determination of the step a) of the method is not carried out, where the procathepsin B being present in the sample is converted into the active form of cathepsin B,

iii) the potentially available activity of cathepsin B coming from procathepsin B in the sample is calculated as a difference value between the first determination i) and the second determination ii).

The difference value calculated in this manner corresponds to the potential cathepsin B activity, which could be obtained from the procathepsin B being present in the sample by converting it into cathepsin B.

The present invention is further explained in the following by means of examples and preferred embodiments.

1) Preparation and supply of the sample
1.a) Blood is coagulated according to standard methods, and after centrifugation serum is obtained which is portioned and filled into Eppendorf tubes and stored at the temperature of liquid nitrogen until the experiments are carried out.
1.b) Tissue homogenate is obtained from tissue samples according to standard methods, then it is portioned, filled into Eppendorf tubes and stored at the temperature of the liquid nitrogen until the measurements is carried out.

2) Conversion of procathepsin B in the sample into the active form of cathepsin B
2.a) By reaction with pepsin:
As the optimal pH-value for the conversion of procathepsin B into the active form of cathepsin B is 4.5, the following experiments are carried out at this pH-value. For this 0.2 ml of the serum sample is diluted 1:1 with acetate buffer (pH=4.5), where porcine pepsin is already dissolved. This solution is incubated at 30° C. for twenty minutes. Afterwards the pH value of the solution is set at 6 for the enzyme assay by means of phosphate buffer.
2.b) By reaction with cathepsin D:
For this conversion also 0.2 ml of the serum sample are diluted 1:1 with acetate buffer to pH 4.5, in which the human cathepsin D is already dissolved. This solution will then be incubated at 37° C. for four hours. Afterwards the pH value of the solution is set at 6 for the enzyme assay by means of phosphate buffer.
2.c) By lowering the pH-value:
A serum sample of 0.2 ml is diluted 1:1 with acetate buffer to pH 4.5 and then incubated at 30° C. for forty minutes. Afterwards the pH value of the solution is set at 6 for the enzyme assay by means of phosphate buffer.

3) How to make “modified” papain having an inactive protelolytic function
3.a) Pre-treatment of the immobilised papain:
Commercially available papainagarose is suspended in 50% glycerol, sodium acetate buffer (0.1 M, pH=4.5) containing 0.05% Na-N₃). Therefore, at first this solution is exchanged against phosphate buffer pH=6. Per enzyme assay 50 μl papain-agarosegel (=100 μl suspension) are employed. This corresponds to 5.5·10⁻¹⁰ Mol papain. Therefore, for 32 determinations 3.2 ml suspension are used. The exchange against phosphate buffer is carried out by a fourfold sequence of addition of buffer, re-suspension, centrifugation and discarding the supernatant.
Papain covalently coupled to cellulose is available on filter paper of a diameter of 110 mm which may be divided into 32 equal segments, whereby each segment has about the same portion of papain as 50 μl papain-agarosegel.
3.b) Reaction of the immobilised papain with methyl methanthiosulfonate (MMTS):
To papain-agarosegel, which is obtained as described above, a solution of phosphate buffer (pH=6) containing 5mM MMTS is added and then re-suspended. For further use this gel is centrifuged and the supernatant discarded.
The cellulose filter to which papain is covalently bound is dipped into a phosphate buffer solution (pH=6) containing 5 mM MMTS and afterwards removed and divided into segments for further use.
3.c) Reaction of immobilised papain with p-mercuribenzoate:
To papain-agarosegel, which is obtained as described above, a phosphate buffer solution (pH=6) containing 0.02 μMol p-mercuribenzoate is added and resuspended. For further use this gel is centrifuged and the supernatant is discarded. In order to remove the excess of reagent a cycle of adding phosphate buffer (pH=6), resuspension, centrifugation and rejection of the supernatant is applied four times.
The cellulose filter, to which the papain is covalently bound, is dipped into a phosphate buffer solution (pH=6) containing 2 mM p-mercuribenzoate and afterwards removed and divided into segments for further use.
3.d) Reaction of the immobilised papain with N-ethylmaleimide
To papain-agarosegel, which is obtained as described above, a phosphate buffer solution (pH=6) containing 2 mM N-ethylmaleimide is added and resuspended. For further use this gel is centrifuged and the supernatant is discarded. In order to remove the excess of reagent a cycle of adding phosphate buffer (pH=6), resuspension, centrifugation and rejection of the supernatant is applied four times.
The cellulose filter, to which the papain is covalently bound, is dipped into a phosphate buffer solution (pH=6) containing 2 mM N-ethylmaleimide and afterwards removed and divided into segments for further use.

4) Removing the free inhibitor from the sample and deinhibition of cathepsin B
4.a) Contacting the sample with immobilised modified papain:
Modified papain immobilised on agarosegel is used as follows: to 160 μl of a serum sample, which is 1:1 diluted with phosphate buffer (pH=6) and in which procathepsin B is already converted into cathepsin B, 50 μl of the modified papain-agarosegel is added in an Eppendorf tube, then the gel is resuspended and after complete deinhibition the reaction vessel is centrifuged at 10000 rpm. From the supernatant 110 μl are transferred into a 0.5 ml reaction vessel for the enzymatic determination of the activity of cathepsin B.

Alternatively, modified papain covalently bound to cellulose is used as follows: 160 μl of a serum sample, which is 1:1 diluted with phospate buffer (pH=6) and in which procathepsin B is already converted into cathepsin B, are contacted in an Eppendorf tube with a piece of cellulose, to which about 5.5·10⁻¹⁰ Mol modified papain is covalently bound, by dipping until complete deinhibition. Subsequently the cellulose portion is removed from the solution. From the deinhibited serum sample 110 μl are transferred into a 0.5 ml reaction vessel for the enzymatic determination of the activity of cathepsin B.
4.b) Contacting the sample with immobilised non-modified papain and subsequently with modified papain
Papain immobilised on agarosegel is used as follows: to 160 μl of a serum sample, which is 1:1 diluted with phospate buffer (pH=6) and in which procathepsin B is already converted into cathepsin B, 50 μl of papain-agarosegel is added in an Eppendorf tube, then the gel is resuspended and the reaction vessel is centrifuged at 10000 rpm for 30 seconds. The supernatant is transferred into a 0.5 ml reaction vessel and 50 μl of the modified papain-agarosegel is added. The gel is resuspended and after complete deinhibition the reaction vessel is centrifuged at 10000 rpm. From the supernatant 110 μl are transferred into a 0.5 ml reaction vessel for the enzymatic determination of the activity of cathepsin B.

Alternatively, non-modified papain covalently bound to cellulose is used as follows: 160 μl of a serum sample, which is 1:1 diluted with phospate buffer (pH 6) and in which procathepsin B is already converted into cathepsin B, are contacted in an Eppendorf tube with a piece of cellulose to which about 5.5·10⁻¹⁰ Mol papain is covalently bound by dipping and subsequently the piece of cellulose is removed from the solution. In this solution sample a further piece of cellulose, to which about 5.5·10⁻¹⁰ Mol modified papain is covalently bound, is dipped and after complete deinhibition removed. Then 110 μl of the sample are transferred into a 0.5 ml reaction vessel for the enzymatic determination of the activity of cathepsin B.

5) Conversion of procathepsin B into cathepsin B by immobilised papain, simultaneous removal of the pool of free inhibitors and subsequently deinhibition of cathepsin B by immobilised modified papain

Papain immobilised on agarosegel is used as follows:

to 160 μl of a serum sample, which is 1:1 diluted with phospate buffer (pH=6), 50 μl of papain-agarosegel is added in an Eppendorf tube, then the gel is re-suspended and the reaction vessel is centrifuged at 10000 rpm for 30 seconds. The supernatant is transferred into a 0.5 ml reaction vessel and 50 μl of the modified papain-agarosegel are added, the gel is resuspended and after complete deinhibition the reaction vessel is centrifuged at 10000 rpm. 110 μl of the supernatant are transferred into a 0.5 ml reaction vessel for the enzymatic determination of the activity of cathepsin B.

Alternatively, after addition of 50 μl papain-agarosegel and resuspension, to the suspension 5 mM MMTS is added and after complete deinhibition the reaction vessel is centrifuged at 10000 rpm. 110 μl of the supernatant are transferred into a 0.5 ml reaction vessel for the enzymatic determination of the activity of cathepsin B.

Non-modified papain covalently bound to cellulose is used as follows:

160 μl of a serum sample, which is 1:1 diluted with phospate buffer (pH 6), are contacted in an Eppendorf tube with a piece of cellulose to which about 5.5·10⁻¹⁰ Mol papain is covalently bound by dipping and subsequently the piece of cellulose is removed from the solution. Into this sample a piece of cellulose, to which about 5.5·10⁻¹⁰ Mol modified papain is covalently bound, is dipped and after complete deinhibition removed. Then 110 μl of the sample are transferred into a 0.5 ml reaction vessel for the enzymatic determination of the activity of cathepsin B.

Alternatively, after the first dipping of the piece of cellulose, to which papain is covalently bound, 5 mM MMTS is added to the serum and after completion of the deinhibition the piece of cellulose is removed from the sample. Then 110 μl of the sample are transferred into a 0.5 ml reaction vessel for the enzymatic determination of the activity of cathepsin B.

6) Measuring the activity of cathepsin B in the sample

To 110 μl of the serum samples, which have been treated as described, in 0.5 ml reaction vessels 110 μl of a solution of the substrate (70 μM Z-Arg-Arg-AFC) are added, the pH-value of which is set at pH=6 by phosphate buffer and which contains 5 mM EDTA and 10 mM DTE. After mixing the samples are incubated for 120 min at 37° C. Subsequently the reaction vessels are cooled down with ice, centrifuged for a short time so as to bring the water condensed at the cap of the reaction vessel back to the solution and then the fluorescence emission is recorded, either on a microtiter plate by means of a fluorescence reader or in a cuvette by means of a fluorimeter. The excitation wavelength is at about 400 nm, and a suitable detection wavelength is at about 505 nm.

The measurement signal is after addition of the artificial cysteine protease specific inhibitor E 64 and of the cathepsin B specific inhibitor CA-074 to the enzyme assay the same as the measuring signal, which is obtained, when the enzyme assay is carried out before withdrawing the inhibitor. Thus evidence is established that the value measured in the enzyme assay after withdrawing the inhibitor relates to cathepsin B which is set free.

1. A method for determination of the potentially available activity of cathepsin B in a biological sample, including the activity of the active form of cathepsin B, the form of cathepsin B which can be activated from the pro-form procathepsin B being present in the sample, and the form of cathepsin B which can be activated and which is inhibited in the sample in its activity by protease inhibitors, with the following steps:
a) procathepsin B being present in the sample is converted into the active form of cathepsin B by means of
a.i) contacting the sample with a first enzyme (proteolytic enzyme) the functionality of which is able to convert procathepsin B into the active form of cathepsin B by proteolytic digestion, or
a.ii) lowering the pH value to a value where procathepsin B is converted into the active form of cathepsin B,
b) depletion of the free protease inhibitor of cathepsin B in the sample or suppression of its inhibitor function and withdrawing the protease inhibitor from the inhibited form of cathepsin B by contacting the sample with a second enzyme (inhibitor binding enzyme) which is able to bind the protease inhibitor of cathepsin B and has a higher affinity to the protease inhibitor than cathepsin B, whereby
b.i) the first enzyme (proteolytic enzyme) and the second enzyme (inhibitor binding enzyme) are the same enzyme provided that the enzyme has the function to convert procathepsin B into the active form of cathepsin B by proteolytic digestion as well as to bind the protease inhibitor for cathepsin B and has a higher affinity to the protease inhibitor than cathepsin B,
or
b.ii) the utilised second enzyme (inhibitor binding enzyme) differs from the first enzyme (proteolytic enzyme), if it is used,
whereby
the second enzyme (inhibitor binding enzyme) is an enzyme which has not a proteolytic activity for degrading cathepsin B, or
the second enzyme (inhibitor binding enzyme) is an enzyme which has a proteolytic activity for degrading cathepsin B, and this proteolytic activity of the second enzyme for degrading cathepsin B is inactivated after a reaction time of step b) in the sample, or the second enzyme is removed from the sample and substituted by an enzyme which has not an activity for degrading cathepsin B but is able to bind the protease inhibitor for cathepsin B and has a higher affinity to the protease inhibitor than cathepsin B,
c) contacting the sample with a substrate for cathepsin B and recording the proteolytic reaction of the substrate catalysed by the protease cathepsin B.

2. the method according to claim 1, wherein the first enzyme (proteolytic enzyme), which has the functionality to convert procathepsin B into the active form of cathepsin B by proteolytic digestion, is a hydrolase without protease activity for degradation of cathepsin B, preferably pepsin or cathepsin D or cathepsin C or thermolysin or pronase, particularly preferred pepsin or Cathepsin D, and that the used second enzyme (inhibitor binding enzyme) differs from the first enzyme (proteolytic enzyme).

3. The method according to claim 2, wherein the used second enzyme (inhibitor binding enzyme) is papain, which has the functionality to bind the protease inhibitor for cathepsin B and has a higher affinity to the protease inhibitor than cathepsin B, whereby the protease activity of the papain for the degradation of cathepsin B after a reaction time is activated in the sample or the papain is removed from the sample and substituted by an enzyme which has not a protease activity for the degradation of cathepsin B but has the functionality to bind the protease inhibitor for cathepsin B and has a higher affinity to the protease inhibitor than cathepsin B.

4. The method according to claim 1, wherein the used first enzyme (proteolytic enzyme) and the second enzyme (inhibitor binding enzyme) are the same enzyme papain, which has the functionality to convert procathepsin B into the active form of cathepsin B by proteolytic digestion as well as to bind the protease inhibitor for cathepsin B and has a higher affinity to the protease inhibitor than cathepsin B, whereby the protease activity of papain for the degradation of cathepsin B is activated in the sample after a reaction time or the papain is removed from the sample and substituted by an enzyme which has not a protease activity for the degradation of cathepsin B but has the functionality to bind the protease inhibitor for cathepsin B and has a higher affinity to the protease inhibitor than Cathepsin B.

5. The method according to claim 3, wherein after a reaction time papain in the sample is converted into a modified papain having an inactivated protease activity for the degradation of cathepsin B, whereby the modified papain lacking the protease activity can be made by
chemical modification of the SH-group of the cysteine in the proteolytic active centre of papain, preferably by reaction of papain with methyl methanthiosulfonate (MMTS), p-mercuribenzoate or AgNO₃ or by addition of N-substituted maleimide.

6. The method according to claim 3, wherein after a reaction time papain in the sample is removed and substituted by a modified papain, which lacks the protease activity for the degradation of cathepsin B but has the functionality to bind the protease inhibitor for cathepsin B and has a higher affinity to the protease inhibitor than cathepsin B, whereby the modified papain lacking the protease activity can be made by
a) chemical modification of the SH-group of the cysteine in the proteolytic active centre of the papain, preferably by reaction of papain with methyl methanthiosulfonate (MMTS), p-mercuribenzoate or AgNO₃ or by addition of N-substituted maleimide, or by
b) site-directed mutation of cysteine in the proteolytic active centre of the papain by another amino acid.

7. The method according to claim 1, wherein the used second enzyme (inhibitor binding enzyme) is modified papain which has the functionality to bind the protease inhibitor for cathepsin B and has a higher affinity to the protease inhibitor than cathepsin B but lacks the functionality to convert procathepsin B into the active form of cathepsin B by proteolytic digestion and lacks the protease activity for the degradation of cathepsin B, whereby the modified papain lacking the protease activity can be made by
a) chemical modification of the SH-group of the cysteine in the proteolytic active centre of the papain, preferably by reaction of papain with methyl methanthiosulfonate (MMTS), p-mercuribenzoate or AgNO₃ or by addition of N-substituted maleimide, or by
b) site-directed mutation of cysteine in the proteolytic active centre of the papain by another amino acid.

8. The method according to claim 1, wherein the inactivation of the protease activity of the second enzyme (inhibitor binding enzyme) for cathepsin B is carried out after a reaction time of step b) of 5 min, preferably after a reaction time of the step b) of 3 min, particularly preferred after a reaction time of the step b) of 1 min.

9. The method according to claim 1, wherein the biological sample is blood, blood plasma, serum or a tissue homogenate.

10. The method according to claim 1, wherein the substrate for cathepsin B comprises a di- or oligopeptide sequence and a fluorophore, which can be cleaved during the proteolytic reaction of the substrate by the protease cathepsin B from the oligopeptide sequence, whereby the fluorophore is preferably 7-amino-4-trifluoromethylcoumarin (AFC) or 7-amino-4-methylcoumarin (AMC).

11. The method according to claim 1, wherein the first enzyme (proteolytic enzyme), preferably papain, is bound covalently or in an adsorptive manner to a carrier, preferably to agarose gel or to cellulose, particularly preferred covalently to cellulose, exceptionally preferred covalently to cellulose obtained chemically or photochemically.

12. The method according to claim 1, wherein step a) of contacting the sample with a first enzyme (proteolytic enzyme), which has the functionality to convert procathepsin B into the active form of cathepsin B by proteolytic digestion, is carried out at a temperature in the range between 4 and 40° C., preferably between 20 and 40° C. and at a pH-value in the range between 1 and 6, preferably between 2 and 5, particularly preferred at 4.5.

13. The method according to claim 1, wherein step b) of contacting the sample with a second enzyme (inhibitor binding enzyme) is carried out at a temperature in the range between 4 to 40° C., preferably between 20 and 40° C. and at a pH-value in the range between 2 and 7, particularly preferred between 4.5 and 6.

14. A method for determination of the pro-form of cathepsin B, procathepsin B, in the sample, wherein in that
i) with a first portion of the biological sample a first determination of the potentially available acitivity of cathepsin B is carried out according to claim 1,
ii) with a second portion of the same biological sample a second determination of the potentially available activity of cathepsin B is carried out according to claim 1, whereby, however, for the second determination the step a) of the method is not carried out, in which the procathepsin B present in the sample is converted into the active form of cathepsin B,
iii) the potentially available activity of cathepsin B coming from procathepsin B in the sample is calculated as a difference value between the first determination i) and the second determination ii).
